(19) United States
(12) Patent Application Publication
(10) Pub. No.: US 2006/0029926 A1
(43) Pub. Date: Feb. 9, 2006

(54) Blocking compositions for immunoassays

(75) Inventors: Jan W. Pawlak, San Jose, CA (US); John E. Bartz, Milpitas, CA (US); Catherine Pawlak, San Jose, CA (US); Patrick M. Sexton, San Diego, CA (US); Charles P. Zahl JR., Mountain View, CA (US); Tilden L. Capen-Frederick, Santa Cruz, CA (US); Victor A. Manneh, Del Mar, CA (US)

Correspondence Address: Laurie A. Axford;Gordon & Rees LLP, Suite 1600, 101 West Broadway, San Diego, CA, 92101, US

(73) Assignee: METRIKA, Inc., Sunnyvale, CA (US)

(21) Appl. No.: 11/077,451

(22) Filed: Mar. 9, 2005

Related U.S. Application Data
(63) Continuation of application No. 09/306,474, filed on May 6, 1999, now abandoned.
(60) Provisional application No. 60/084,438, filed on May 6, 1998.

(51) Int. Cl. C12Q 1/00; G01N 33/558

(52) U.S. Cl. 435/4; 436/514

(57) ABSTRACT

Compositions and processes for qualitative or quantitative one-step, two-site, tag/anti-tag or competitive non-bibulous lateral flow (immunochromatographic) assays for analytes in body fluids including chemically modified proteins as blocking and/or dispersing agents in conjunction with additives eliminating non-specific interference with the detection agents and/or binding partner caused by endogenous polypeptide constituents. Composition of the chemically modified proteins can be albumins. Composition of the chemically modified albumins can have altered charge and/or molecular weight. Process for composition of the chemically modified proteins can be prepared by modification of the nucleophilic groups. The chemical modification of nucleophilic groups in albmins can be introduced by anhydrides, alkyl acetimidates, methylating and/or cross-linking agents. The additives eliminating non-specific interference can be chemically modified albumins, heterophilic blockers and chaotropic agents.

### FIELD OF THE INVENTION

Compositions and processes for qualitative or quantitative one-step, two-site, tag/anti-tag or competitive non-bibulous lateral flow (immunochromatographic) assays for analytes in body fluids including chemically modified proteins as blocking and/or dispersing agents in conjunction with additives eliminating non-specific interference with the detection agents and/or binding partner caused by endogenous polypeptide constituents.

Composition of the chemically modified proteins can be albumins.

Composition of the chemically modified albumins can have altered charge and/or molecular weight.

Process for composition of the chemically modified proteins can be prepared by modification of the nucleophilic groups. The chemical modification of nucleophilic groups in albmins can be introduced by anhydrides, alkyl acetimidates, methylating and/or cross-linking agents.

The additives eliminating non-specific interference can be chemically modified albumins, heterophilic blockers and chaotropic agents.

### BACKGROUND OF THE INVENTION

Compositions and processes designed for one-step lateral flow (immunochromatographic) immunoassays described in prior art (e.g. EP 0 284 232 A1, EP 0 291 194 B1, U.S. Pat. No. 4,861,711, EP 0 299 428 B1, U.S. Pat. No. 5,591,645, U.S. Pat. No. 5,712,170, PCT WO 91/12336, PCT WO 88/08534, PCT WO 94/23300) do not provide for specific and sensitive immunoasays using whole blood/serum specimens in particular.

### SUMMARY OF THE INVENTION

Compositions and processes for qualitative or quantitative one-step, two-site, tag/anti-tag or competitive non-bibulous lateral flow (immunochromatographic) assays for analytes in body fluids including chemically modified proteins as blocking and/or dispersing agents in conjunction with additives eliminating non-specific interference with the detection agents and/or binding partner caused by endogenous polypeptide constituents.

Composition of the chemically modified proteins can be albumins.

Composition of the chemically modified albumins can have altered charge and/or molecular weight.

Process for composition of the chemically modified proteins can be prepared by modification of the nucleophilic groups. The chemical modification of nucleophilic groups in albmins can be introduced by anhydrides, alkyl acetimidates, methylating and/or cross-linking agents.

The additives eliminating non-specific interference can be chemically modified albumins, heterophilic blockers and chaotropic agents.

### DESCRIPTION OF THE PREFERRED EMBODIMENTS

### EXAMPLE 1

To activate the labeling beads, one ml of 0.403 um Dark Blue latex particles (MPs; P/N 10012; Metrika, Inc., Sunnyvale, Calif.) at 10% (w/v) solids are combined with 1 ml of 0.5 M MES buffer (pH 6.0), 5.5 ml of deionized H₂O, 2.3 ml of 50 mg of N-hydroxysuccinimide (NHS; Product # 24500; Pierce Chemical Company, Rockford, Ill.) per ml deionized H₂O and 0.2 ml of 5 mg of 1-ethyl-3-(3-dimethylamino-propyl)carbodiimide hydrochloride (EDC; Product # 22980; Pierce Chemical Company) in deionized H₂O. The resultant mixture is sonicated on ice for 40 seconds and then allowed to react on a shaker at RT for 30 minutes. The activated MPs are then centrifuged at 10° C. at 10,000×g and washed three times with cold 50 mM MES buffer (pH 6.0) by resuspension and centrifugation cycles. In a typical procedure, the final pellet of MPs is suspended in 3.666 ml of 50 mM MES buffer (pH 6.0), a 5 ml mixture of 0.6-1.2 mg antigen-specific antibody such as e.g. goat anti-Troponin I peptide 3 antibody (Product code # G-129-C; BiosPacific/Fortron Bio Science Inc., Emeryville, Calif., or Product # 9099A; HTI Bio-Products, Inc., Ramona, Calif.) and 2.5 mg of mouse IgG (cat. I-8765; Sigma Chemical Company, St. Louis, Mo.) in the same buffer are added with mixing, followed by an addition of 5 ml of 0.1 M borate buffer (pH 8.5). The mixture is allowed to incubate at RT for 2 hr and then centrifuged as described above. Subsequently, 10 ml of 50 mM borate buffer (pH 8.5) containing 5 mM ethanolamine (cat. # E-9508; Sigma Chemical Company) is added to the pellet, MPs are suspended, incubated at RT for 30 min, and the suspension is centrifuged as described above.

The remaining hydrophobic sites on MPs are then blocked with FSG blocking solution composed of 0.1% (w/v) of fish skin gelatin (FSG; cat. # G-7765; Sigma Chemical Company) in 50 mM borate buffer (pH 8.5) at RT for 30 min. The MPs blocked with FSG are centrifuged as described above and suspended in 0.2 M EPPS buffer (pH 8.0) containing 0.5% (w/v) of FSG, 0.5% (w/v) of Hammarsten casein (Product # 440203H; BDH Laboratory Supplies, Poole, England), 0.5% (v/v) of Tween 20 (cat. # P-1379; Sigma Chemical Company) and 0.01% (w/v) of NaN₃.

To prepare the capture zone membrane, nitrocellulose having a pore size of >5 um (Product # AE98; Schleicher and Schuell, Keene, N.H.) is affixed to an XY-plotter table. A Streptavidin-BSA (bovine serum albumin) capture band is dispensed in a 2.0 mm zone at the distal end of the nitrocellulose membrane using Streptavidin-BSA conjugate (e.g. L/N 97-02-154; Metrika, Inc.) at 0.85-2.0 mg/ml. The solution is dispensed with an IVEK Digispense (IVEK Corporation, Springfield, Vt.) dispensing system. After air drying at 45° C., the membrane is placed into a tray containing the membrane blocking solution for 20 minutes at RT. The membrane blocking solution comprises of 0.2-0.35% (w/v) of poly(vinyl alcohol) (PVA; 80% hydrolyzed; Av. Mol. Wt. 9,000-10,000; Cat. # 36,062-7; Aldrich Chemical Company, Inc., Milwaukee, Wis.), 0.5% (v/v) BSA (Pentex code # 82-045-1; Bayer Corporation, Kankakee, Ill.) and 0.1% (w/v) of FSG. The membrane is removed and blotted for 5 minutes. The membrane is air dried at 45° C. for 5 minutes, and then placed at less than 5.0% RH overnight. Processed capture membranes remain at less than 5.0% RH until assembly.

Serum samples were drawn at Metrika, Inc. from the twenty-four apparently healthy asymptomatic donor volunteers. The specimens were then analyzed by Behring Opus Plus Troponin I Reference Quantitative Assay (Behring cat # 703-050 and 703-006) and/or by Dade Stratus® Cardiac Troponin-I Fluorometric Enzyme Assay (Dade cat # B5700-64) and they were found to be negative for Troponin I.

For “wet” assays for Troponin I, a 14×100 mm strip of the capture zone membrane is affixed centrally on an adhesive opaque strip. The opaque backing is a 23×100 mm strip of ARCare mylar made adhesive with 3M 9502. The absorbent—which is a 10×100 mm rectangle of Whatman 31ET cellulose paper (Whatman, Inc., Fairfield, N.J.)—is affixed distal to the capture zone pad with 0.5 mm overlap. The sample zone pad composed of 7×100 mm cellulose acetate (Part # 12301R101X50M; Sartorius Corporation, Santa Clara, Calif.) is then placed next to the capture zone membrane with 0.5 mm overlap.

Ten-fold concentrated STB for “wet” assays is comprised of 0.5 M EPPS buffer (pH 8.0) supplemented with 6.25% (v/v) of Tween-20 (cat. P-1379; Sigma Chemical Company), 2% (w/v) of BSA and 0.1% (w/v) of NaN₃. In “wet” assays, 9 μl of specimen sample is mixed sequentially in a test tube with 1 μl of 10-fold concentrated stock of STB, 1 μl of biotinylated F(ab′)₂ fragment of anti-TnI antibody (e.g. mouse monoclonal antibody against Troponin I; Product # TR1-7F83; Dako Corporation, Carpinteria, Calif.; or goat anti-Troponin I peptide 69-80; Code # 9099A, HTI Bio-Products, Inc., Ramona, Calif.) and 1 μl of labeling MPs prepared at 0.5% (w/v) solids. Subsequently, the “wet” assay strip assembled as just described is placed into the tube, allowed to develop for 7 min, then removed and the intensity of the band is measured with a Gretag reflectance densitometer (Model #D19C; Gretag Color Control Systems, Regensdorf, Switzerland). Increasing values from the Gretag instrument indicate increasing color intensity, which corresponds to increasing analyte concentration.

As shown in Table 1, eleven out of twenty-four serum specimens (46%) obtained from Troponin I-negative donors gave false positive results in the lateral flow (immunochromatographic) assay.

TABLE 1
Negative Serum Samples Tested by Metrika TnI Assay

| | Gretag Density | False Positive |
|---|---|---|
| Donor # | Units (GDU) | Result? |
| Neg Bovine | 0.16 | No |
| Calibrator | | |
| 1 | 0.97 | Yes |
| 2 | 0.42 | Yes |
| 3 | 0.26 | No |
| 4 | 0.21 | No |
| 5 | 0.22 | No |
| 6 | 0.20 | No |
| 7 | 0.20 | No |
| 8 | 0.29 | No |
| 9 | 0.30 | No |
| 10 | 0.59 | Yes |
| 11 | 0.24 | No |
| 12 | 0.24 | No |
| 13 | 0.92 | Yes |
| 14 | 0.98 | Yes |
| 15 | 0.29 | No |
| 16 | 0.69 | Yes |
| 17 | 0.30 | No |
| 18 | 0.68 | Yes |
| 19 | 0.28 | No |
| 20 | 0.27 | No |
| 21 | 0.77 | Yes |
| 22 | 0.45 | Yes |
| 23 | 1.17 | Yes |
| 24 | 1.01 | Yes |

### EXAMPLE 2

In order to replace the +ve charge on —NH⁺₃ by a −ve charge on the free —COO⁻ of the maleyl group, maleic anhydride was reacted with BSA under mildly alkaline conditions. Thus 300 mg of BSA was dissolved at room temperature (RT) in 10 ml of 0.2 M carbonate buffer (pH 8.73), and 122.6 mg of maleic anhydride (cat. # M18-8; Aldrich Chemical Company, Milwaukee, Wis.) dissolved in 0.5 ml of anhydrous dimethylformamide (DMF; cat. # 27,054-7; Sigma Aldrich Chemical Company) was added with stirring. It should be noted that under those conditions, maleic anhydride also reacts with —SH groups forming stable derivatives (cf. N-ethylmaleimide). After for 2 hours, 45.3 μl of ethanolamine (cat. # 11,016-7; Aldrich Chemical Company) was added and allowed to react at RT for 30 min. The resultant reaction mixture was buffer exchanged into 25 mM Tris buffer (pH 8.0) containing 0.1% (w/v) NaN₃ using Sephadex G-25 Fine resin (cat. # G-25-80; Sigma Chemical Company, St. Louis, Mo.) to a final concentration of 10 mg/ml.

Acetic anhydride (AA) is a reagent of choice for acetylating protein amino groups. At neutral or mildly alkaline pH (7-9.5), AA reacts with unprotonated α- and ε-NH₂ groups rendering them electrically neutral. Thus, 2.1 g of BSA was dissolved at RT in 70 ml of 0.2 M carbonate buffer (pH 8.7) and 825.7 μl of acetic anhydride (cat. # A-64-4; Sigma Chemical Company) was added with stirring. After for 2 hours, 45.3 μl of ethanolamine was added and allowed to react at RT for 30 min. The resultant reaction mixture was buffer exchanged into 25 mM Tris buffer (pH 8.0) containing 0.1% (w/v) NaN₃ using Sephadex G-25 Fine resin to a final concentration of 10 mg/ml.

Succinic anhydride (SA) reacts preferentially with—NH₂ groups at pH 7-9, converting +ve charge on amino group to a −ve charge; succinyl half-amide derivatives are more stable than maleyl derivatives. Similarly, 150 mg of BSA was dissolved at RT in 5 ml of 0.2 M carbonate buffer (pH 8.7) and 62.56 mg of succinic anhydride (cat. # S-7626; Sigma Chemical Company) dissolved in 250 μl of anhydrous DMF was added with stirring. After for 2 hours, 22.6 μl of ethanolamine was added and allowed to react at RT for 30 min. The resultant reaction mixture was buffer exchanged into 25 mM Tris buffer (pH 8.0) containing 0.1% (w/v) NaN₃ using Sephadex G-25 Fine resin to a final concentration of 10 mg/ml.

Alkyl (e.g. methyl or ethyl) acetimidates react with α- and ε-NH₂ groups on proteins at pH 7-10 forming amidine derivatives that retain +ve charge of original amino group, with pKₐ of amidine being somewhat higher than that of —NH⁺₃. Here, 150 mg of BSA was dissolved at RT in 5 ml of 0.2 M carbonate buffer (pH 8.7), and 77.25 mg of ethyl acetimidate HCl (cat. # E-3880; Sigma Chemical Company) or 68.50 mg of methyl acetimidate HCl (cat. # M-3880; Sigma Chemical Company)—either dissolved in 250 μl of deionized water—was added with stirring. After 2 hours, 22.6 μl of ethanolamine was added and allowed to react at RT for 30 min. The resultant reaction mixture was buffer exchanged into 25 mM Tris buffer (pH 8.0) containing 0.1% (w/v) NaN₃ using Sephadex G-25 Fine resin to a final concentration of 10 mg/ml.

Low molecular weight product: 990 mg of BSA was dissolved at RT in 30 ml of 0.2 M carbonate buffer (pH 8.7), 242 μl of formaldehyde (37.3% solution; cat. G-5250; Sigma Chemical Company) was added with stirring, followed by an addition of 3.33 ml of 1M sodium cyanoborohydride (cat. # 15-615-9; Aldrich Chemical Company) dissolved in the same buffer. After 2 hours, the resultant reaction mixture was buffer exchanged into 25 mM Tris buffer (pH 8.0) containing 0.1% (w/v) NaN₃ using Sephadex G-25 Fine resin to a final concentration of 10 mg/ml.

High molecular weight product: 57.6 g of BSA was dissolved at RT in 960 ml of 0.2 M carbonate buffer (pH 8.7), and 3.272 ml of glutaraldehyde (Grade I; 25% aqueous solution; cat. #G-5882; Sigma Chemical Company) was added with stirring, followed by an addition of 96 ml of 1M sodium cyanoborohydride dissolved in the same buffer. After 4 hours, 104 ml of formaldehyde was added with stirring and allowed to react overnight at RT. The reaction was terminated by adding 106 ml of ethanolamine and allowing to react at RT for 30 min. The resultant mixture was exchanged into 50 mM Tris buffer (pH 8.0) containing 0.1% (w/v) NaN₃ using Sephadex G-25 Fine resin to a final concentration of 10 mg/ml.

To activate the labeling beads, one ml of 0.403 um Dark Blue latex particles (MPs; P/N 10012; Metrika, Inc) at 10% (w/v) solids are combined with 1 ml of 0.5 M MES buffer (pH 6.0), 5.5 ml of deionized H₂O, 2.3 ml of 50 mg of NHS (Product #24500; Pierce Chemical Company) per ml deionized H₂O and 0.2 ml of 5 mg of EDC (Product # 22980; Pierce Chemical Company) in deionized H₂O. The resultant mixture is sonicated on ice for 40 sec and then allowed to react on a shaker at RT for 30 min. The activated MPs are then centrifuged at 10° C. at 10,000×g and washed three times with cold 50 mM MES buffer (pH 6.0) by resuspension and centrifugation cycles. In a typical procedure, the final pellet of MPs is suspended in 3.666 ml of 50 mM MES buffer (pH 6.0), a 5 ml mixture of 0.6-1.2 mg antigen-specific antibody such as e.g. goat anti-Troponin I peptide 3 antibody (Product code # G-129-C; BiosPacific/Fortron Bio Science Inc., or Product # 9099A; HTI Bio-Products) and 2.5 mg of mouse IgG (cat. 1-8765; Sigma Chemical Company) in the same buffer are added with mixing, followed by an addition of 5 ml of 0.1 M borate buffer (pH 8.5). The mixture is allowed to incubate at RT for 2 hr and then centrifuged as described above. Subsequently, 10 ml of 50 mM borate buffer (pH 8.5) containing 5 mM ethanolamine is added to the pellet, MPs are suspended, incubated at RT for 30 min, and the suspension is centrifuged as described above. The remaining hydrophobic sites on MPs are then blocked with either (i) FSG blocking solution composed of 0.1% (w/v) of FSG in 50 mM borate buffer (pH 8.5) at RT for 30 min or (ii) chemically modified BSA solutions described above. The MPs blocked with FSG are centrifuged as described above and suspended in 0.2 M EPPS buffer (pH 8.0) containing 0.5% (w/v) of FSG, 0.5% (w/v) of Hammarsten casein, 0.5% (v/v) of Tween 20 and 0.01% (w/v) of NaN₃. The MPs blocked with chemically modified BSA solution are centrifuged as described above and suspended in the same solution at 10 mg chemically modified BSA per ml.

To prepare passively coated labeling beads, 0.50 ml of 0.403 um Dark Blue latex particles at 2.5% (w/v) solids are combined with 0.50 ml of the respective labeling antibody at 1.0 mg/ml in 25 mM Tris buffer (pH 8.0) containing 0.1% (w/v) NaN₃. The solution is allowed to react passively on an orbital rotator at RT overnight. After centrifugation at 10,000 rpm for 5 minutes, the supernatant is aspirated. The pellet is resuspended in chemically modified BSA solutions described above and MPs are allowed to block for one hour at RT on an orbital rotator. After centrifugation at 10,000 rpm for 5 minutes, the supernatant is aspirated. The pellet is resuspended in chemically modified BSA solution (10 mg/ml) to a final particle concentration of 1.0% (w/v) solids.

To prepare the alternate capture zone membranes, nitrocellulose having a pore size of >5 μm is affixed to an XY-plotter table. A Streptavidin-BSA capture band is dispensed in a 2.0 mm zone at the distal end of the nitrocellulose membrane using Streptavidin-BSA conjugate at 0.85-2.0 mg/ml. The solution is dispensed with an IVEK Digispense dispensing system. After air drying at 45° C., the membrane is placed into a tray containing the membrane blocking solution for 20 minutes at RT. The control membrane blocking solution comprises of 0.2-0.35% (w/v) of PVA. The alternate membrane blocking solutions are chemically modified BSA solutions at 10 mg/ml described above. The membranes are then removed and blotted for 5 minutes. The membranes are air dried at 45° C. for 5 minutes, and then placed at less than 5.0% RH overnight. Processed capture membranes remain at less than 5.0% RH until assembly.

For alternate “wet” assays for Troponin I, a 14×100 mm strip of the capture zone membrane is affixed centrally on an adhesive opaque strip. The opaque backing is a 23×100 mm strip of ARCare mylar made adhesive with 3M 9502. The absorbent—which is a 10×100 mm rectangle of Whatman 31ET cellulose paper—is affixed distal to the capture zone pad with 0.5 mm overlap. The sample zone pad composed of 7×100 mm cellulose acetate is then placed next to the capture zone membrane with 0.5 mm overlap.

An alternate Sample Treatment Buffer (STB) for wet assays is “Control” ten-fold concentrated STB for “wet” assays comprised of 0.466 M EPPS buffer (pH 8.0) supplemented with 1% (w/v) of BSA, 5% (v/v) of Tween-20 and 0.1% (w/v) of NaN₃. To test “alternate” STBs, the following stock solutions were prepared: (i) 4 M urea (cat. # U-5128; Sigma Chemical Company) in 0.466 M EPPS buffer (pH 8.0) containing 0.1% (w/v) of NaN₃; (ii) 5 mg/ml of heterophilic IgG block; (iii) 5-6 mg/ml of goat IgG (cat. # I-5256; Sigma Chemical Company) in 25 mM Tris buffer (pH 8.0) containing 0.1% (w/v) NaN₃; and (iv) 10.4 mg/ml of non-specific Mouse IgG (Technical Grade; cat. # I-5256; Sigma Chemical Company) in the same buffer.

In “wet” assays, 9-10 μl of specimen sample is mixed sequentially in a test tube with 1 μl of 10-fold concentrated stock of either “control” or “alternate” STB, 1 μl of biotinylated F(ab′)₂ fragment of anti-TnI antibody (e.g. mouse monoclonal antibody against Troponin I or goat anti-Troponin I peptide 69-80), and 1 μl of labeling MPs prepared at 0.5% (w/v) solids,. Subsequently, the “wet” assay strip assembled as just described is placed into the tube, allowed to develop for 7 min, then removed and the intensity of the band is measured with a Gretag reflectance densitometer. Increasing values from the Gretag instrument indicate increasing color intensity, which corresponds to increasing analyte concentration.

Serum samples were drawn at Metrika, Inc. from 3 previously identified (Example 1) volunteers whose samples caused severe (donor #1), significant (donor #2) and no (donor #3) interference in the Troponin I lateral flow assay. The specimens were then analyzed by Behring Opus Plus Troponin I Reference Quantitative Assay and/or by Dade Stratus® Cardiac Troponin-I Fluorometric Enzyme Assay, and they were found to be negative for Troponin I.

Subsequently, an Opus TnI calibrator (cat. #703-006; Behring Diagnostics Inc., Westwood, Mass.) was used as an exogenous source of Troponin I which was added to the serum specimens.

Table 2 shows the effectiveness of different blocking reagents at eliminating false positives (FP) and microparticle aggregation caused by serum from Donor #1. None of the blocking reagents tested eliminated false positives or microparticle aggregation.

TABLE 2
Effect of Different Blocks on False Positives (FP) and
Microparticle Aggregation Caused by Serum from Donor #1

| | |
|---|---|
| Blocking Reagent | Result |

| | | |
|---|---|---|
| Maleic BSA | Aggregation | FP |
| Succinylated BSA | Aggregation | FP |
| Diethylimidated BSA | Aggregation | FP |
| Dimethylimidated BSA | Aggregation | FP |
| Bovine IgG | Aggregation | FP |
| PVA | Aggregation | FP |
| Acetylated BSA | Aggregation | FP |

Table 3 shows the effectiveness of different sample treatment buffer (STB) components at eliminating false positives (FP) and microparticle aggregation (Agg) in serum from three donors and a TnI serum calibrator. The results demonstrate that adding Heteroblock to the STB formulation eliminates FP and Agg in samples negative for TnI. However, when these same samples are spiked with 14.7 ng/mL TnI the microparticles badly aggregate, even in the presence of Heteroblock. This aggregation is resolved by the addition of urea to the STB+Heteroblock mixture. Thus a STB formulation which includes Heteroblock and urea solves the problem of false positives and microparticle aggregation.

TABLE 3
Effect of Sample Treatment Buffer Upon False
Positives (FP) and Microparticle Aggregation (Agg)

| Calibrator | | Donor #1 | | Donor #2 | | Donor #3 | |
|---|---|---|---|---|---|---|---|
| FP? | Agg? | FP? | Agg? | FP? | Agg? | FP? | Agg? |

With LBB System (cf. Example 1)

Negative
Sample

| | | | | | | | | |
|---|---|---|---|---|---|---|---|---|
| STB | no | no | | | yes | little | | |
| STB + | no | no | yes | little | little | little | no | no |
| Goat IgG | | | | | | | | |
| STB + | no | no | little | little | little | little | no | no |
| Mouse IgG | | | | | | | | |
| STB + | no | no | no | no | no | no | no | no |
| Heteroblock | | | | | | | | |
| 14.7 ng/mL | | | | | | | | |
| Sample | | | | | | | | |
| STB | | no | | | | | | |
| STB + | | no | | | | | | |
| Goat IgG | | | | | | | | |
| STB + | | no | | | | | | |
| Mouse IgG | | | | | | | | |
| STB + | | no | | yes | | yes | | yes |
| Heteroblock | | | | | | | | |
| STB + | | | | no | | | | |
| Het + Urea | | | | | | | | |

With AcBSA System (cf. Alternate STB of this example)

Negative
Sample

| | | | | | | | | |
|---|---|---|---|---|---|---|---|---|
| STB + | no | no | no | no | no | no | no | no |
| Heteroblock | | | | | | | | |
| STB + | no | no | no | no | no | no | no | no |
| Het + Urea | | | | | | | | |
| 14.7 ng/mL | | | | | | | | |
| Sample | | | | | | | | |
| STB + | | no | | yes | | yes | | yes |
| Heteroblock | | | | | | | | |
| STB + | | no | | no | | no | | no |
| Het + Urea | | | | | | | | |

### EXAMPLE 3

To activate the labeling beads, one ml of 0.403 um Dark Blue latex particles at 10% (w/v) solids are combined with 1 ml of 0.5 M MES buffer (pH 6.0), 5.5 ml of deionized H₂O, 2.3 ml of 50 mg of NHS per ml deionized H₂O and 0.2 ml of 5 mg of EDC in deionized H₂O. The resultant mixture is sonicated on ice for 40 sec and then allowed to react on a shaker at RT for 30 min. The activated MPs are then centrifuged at 10° C. at 10,000×g and washed three times with cold 50 mM MES buffer (pH 6.0) by resuspension and centrifugation cycles. In a typical procedure, the final pellet of MPs is suspended in 3.666 ml of 50 mM MES buffer (pH 6.0), a 5 ml mixture of 0.6-1.2 mg antigen-specific antibody such as e.g. goat anti-Troponin I peptide 3 antibody (Product code # G-129-C; BiosPacific/Fortron Bio Science Inc.) and 2.5 mg of mouse IgG in the same buffer is added with mixing, followed by an addition of 5 ml of 0.1 M borate buffer (pH 8.5). The mixture is allowed to incubate at RT for 2 hr and then centrifuged as described above. Subsequently, 10 ml of 50 mM borate buffer (pH 8.5) containing 5 mM ethanolamine is added to the pellet, MPs are suspended, incubated at RT for 30 min, and the suspension is centrifuged as described above. The remaining hydrophobic sites on MPs are then blocked with acetylated BSA solutions (10 mg/ml) described above; the pellet is resuspended to a final particle concentration of 0.5% (w/v) solids.

To prepare the capture zone membranes, nitrocellulose having a pore size of >5 um (Part # 11301; Sartorius Corporation) is affixed to an XY-plotter table. A Streptavidin-BSA capture band is dispensed in a 2.0 mm zone at the distal end of the nitrocellulose membrane using Streptavidin-BSA conjugate at 2.57 mg/ml. The solution is dispensed with an IVEK Digispense dispensing system. After air drying at 45° C., the membrane is placed into a tray containing the membrane blocking solution comprised of acetylated BSA solution at 10 mg/ml for 20 minutes at RT. The membranes are then removed and blotted for 5 minutes. The membranes are air dried at 45° C. for 5 minutes, and then placed at less than 5.0% RH overnight. Processed capture membranes remain at less than 5.0% RH until assembly.

For “wet” assays for Troponin I, a 14×100 mm strip of the capture zone membrane is affixed centrally on an adhesive opaque strip. The opaque backing is a 23×100 mm strip of ARCare mylar made adhesive with 3M 9502. The absorbent—which is a 10×100 mm rectangle of Whatman 31ET cellulose paper—is affixed distal to the capture zone pad with 0.5 mm overlap. The sample zone pad composed of 7×100 mm cellulose acetate is then placed next to the capture zone membrane with 0.5 mm overlap. Serum samples were collected at The St. Joseph's Hospital, Stockton, Calif. from patients suspected of myocardial infarction, and the samples were analyzed at the site for Total-CK, CK-MB and Myoglobin levels. Upon receiving, the specimens were analyzed at Metrika, Inc. for Troponin I levels by Behring Opus Plus Troponin I Reference Quantitative Assay, and the respective values were assigned. Table 4 summarizes the just mentioned data.

A Sample Treatment Buffer (STB) for “wet” assays is “Control” 10-fold concentrated STB for “wet” assays comprised of 0.5 M Tris buffer (pH 8.0) supplemented with 10 mg/ml of acetylated BSA, 5% (v/v) of Tween-20, 0.3-0.6 mg/ml of heterophilic IgG block Heteroblock; P/N 70506; Omega Biologicals Inc., Bozeman, Mont.), and 0.1% (w/v) of NaN₃. The following “alternate” STBs were prepared: (i) “control” STB plus 0.5 M Urea; (ii) STB described in (i) supplemented with 5% (v/v) of bovine serum (cat. # B 8655; Sigma Chemical Company); (iii) 50 mM Tris (pH 8.0) containing 10 mg/ml of acetylated BSA, 5% (v/v) bovine serum, 10 μg/ml of purified rabbit skeletal muscle Troponin C (TnC; cat. # T4924, Scripps Laboratories), 150 mM CaCl₂, 0.3-0.6 mg/mL Heteroblock, and 0.1% (w/v) NaN₃; (iv) STB described in (i) supplemented with 10 μg/ml of purified rabbit skeletal muscle Troponin C and 150 mM CaCl₂; and (v) STB described in (iv) supplemented with 5% bovine serum.

In “wet” assays, 9-10 μl of specimen sample is mixed sequentially in a test tube with 1 μl of 10-fold concentrated stock of either “control” or “alternate” STB, 1 μl of biotinylated F(ab′)₂ fragment of goat anti-TnI peptide 69-80 antibody (Code # 9099A, HTI Bio-Products, Inc., Ramona, Calif.) and 1 μl of labeling MPs prepared at 0.5% (w/v) solids. Subsequently, the “wet” assay strip assembled as just described is placed into the tube, allowed to develop for 7 min, then removed and the intensity of the band is measured with a Gretag reflectance densitometer. Increasing values from the Gretag instrument indicate increasing color intensity, which corresponds to increasing analyte concentration.

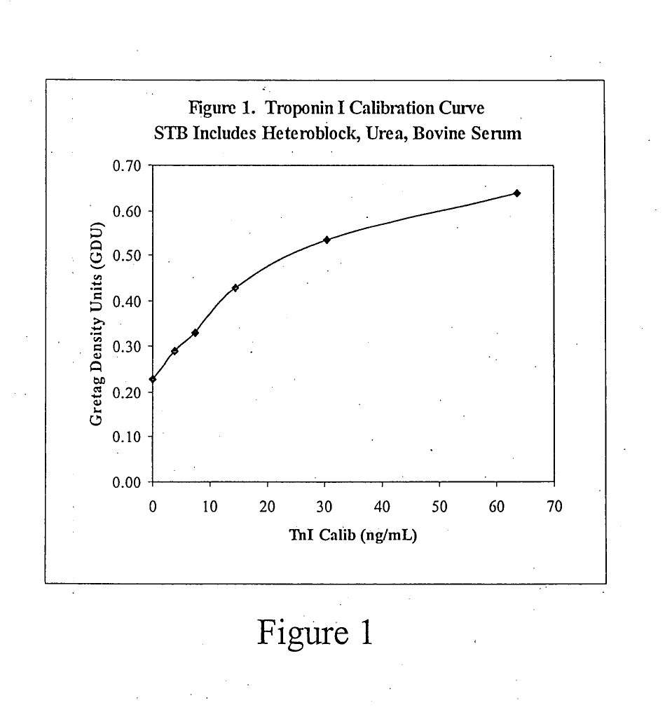

FIG. 1 shows a typical Metrika TnI calibration curve, using STB (ii) including Heteroblock, urea and bovine serum.

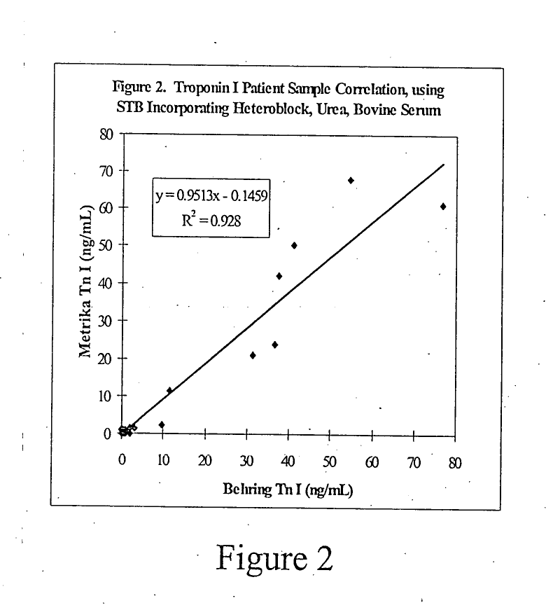

Table 4 shows the results of 33 patient samples tested with the Metrika TnI assay and the Behring TnI assay. The Metrika patient samples were tested using STB formulation (ii) including Heteroblock, urea and bovine serum. The raw reflectance density for each sample test strip is recorded as Metrika GDU. This density is converted to clinical ng/mL TnI using the calibration curve in FIG. 1, and this clinical value is recorded in the Metrika TnI (ng/mL) column. The Behring Opus TnI assay was used to determine reference values. FIG. 2 shows the Metrika and Behring TnI patient sample correlation for the samples in Table 4. Correlation to the Behring TnI assay is 0.928 with a slope of 0.95.

TABLE 4
Troponin I Patient Sample Results

| Patient | Metrika | Metrika | Behring |
|---|---|---|---|
| Sample # | GDU | TnI (ng/mL) | TnI (ng/mL) |
| 46 | 0.49 | 23.8 | 36.6 |
| 47 | 0.22 | 0.0 | 0.0 |
| 48 | 0.40 | 11.5 | 11.5 |
| 49 | 0.28 | 2.4 | 9.8 |
| 50 | 0.47 | 21.2 | 31.3 |
| 51 | 0.23 | 0.0 | 0.0 |
| 52 | 0.24 | 0.9 | 0.0 |
| 53 | 0.57 | 42.2 | 37.5 |
| 54 | 0.20 | 0.0 | 0.0 |
| 55 | 0.21 | 0.0 | 0.0 |
| 56 | 0.60 | 50.5 | 41.2 |
| 57 | 0.23 | 0.0 | 0.0 |
| 59 | 0.23 | 0.0 | 0.8 |
| 60 | 0.22 | 0.0 | 0.0 |
| 61 | 0.22 | 0.0 | 0.0 |
| 62 | 0.22 | 0.0 | 2.0 |
| 63 | 0.24 | 1.0 | 0.0 |
| 64 | 0.64 | 61.2 | 76.6 |
| 65 | 0.23 | 0.8 | 0.0 |
| 66 | 0.23 | 0.0 | 0.0 |
| 67 | 0.23 | 0.8 | 0.5 |
| 68 | 0.66 | 68.0 | 54.6 |
| 69 | 0.22 | 0.0 | 0.5 |
| 70 | 0.21 | 0.0 | 0.0 |
| 71 | 0.24 | 0.9 | 0.9 |
| 72 | 0.22 | 0.0 | 0.0 |
| 73 | 0.24 | 0.9 | 0.0 |
| 74 | 0.23 | 0.0 | 0.0 |
| 75 | 0.21 | 0.0 | 0.0 |
| 76 | 0.26 | 1.5 | 3.0 |
| 77 | 0.25 | 1.3 | 1.9 |
| 78 | 0.22 | 0.0 | 0.0 |
| 79 | 0.22 | 0.0 | 0.0 |

### EXAMPLE 4

To verify the effectiveness of the chemically modified BSA blocking solutions in conjunction with the appropriate selection of STB, the quantitative hCG assay was constructed in a lateral flow format.

The sample receiving zone is prepared from Ahlstrom 1281 (Ahlstrom Filtration Inc., Mt. Holly Springs, Pa.) material. The material is saturated with a blood separating solution at 45 ul/cm² containing 2.5 mg/ml rabbit anti-human red blood cells (Code 209-4139; Rockland Immunochemicals, Gilbertsville, Pa.) antibody diluted in acetylated bovine serum albumin (AcBSA) or methylated BSA (mBSA) prepared at 10 mg/ml. The membrane is frozen at −70° C. for at least one hour and then lyophilized in a Virtis Genesis (Virtis, Gardiner, N.Y.) overnight. The treated sample receiving zone is cut into 7.0×7.0 mm squares and stored at less than 5.0% relative humidity (RH) until assembly.

The sample treatment zone is prepared from Ahlstrom 1281 material. The material is treated with a sample treatment buffer (STB) at 45 ul/cm². STB is composed of 0.5M Sodium Perchlorate in 50 mM Tris buffer, 2.0 mg/ml non-specific Mouse IgG (P/N 9902; Intergen Company, Milford, Mass.), and 1.67 mg/ml heterophilic IgG block. The pad of Ahlstrom 1281 is frozen at −70° C. for at least one hour. The Ahlstrom material is lyophilized in the Virtis Genesis overnight. The sample treatment zone is then cut into 3.5×3.0 mm rectangles and stored at less than 5.0% RH until assembly.

To prepare the labeling beads, 0.50 ml of 0.36 um blue latex particles (P/N LC9786; Emerald Diagnostics Inc., Eugene, Oreg.) at 2.5% solids is combined with 0.50 ml Monoclonal Anti-hCG (clone #5008; Oy Medix Biochemica Ab, Kauniainen, Finland) antibody in 25 mM Tris buffer at 1.0 mg/ml. The solution is allowed to react passively on an orbital rotator at room temperature (RT) overnight. After centrifugation at 10000 rpm for 5 minutes, the supernatant is aspirated. The pellet is resuspended manually with highly polymerized bovine serum albumin (polyBSA) (P/N 99-012-5; Bayer Corporation, Kankakee, Ill.) solution (10 mg/ml). The particles are allowed to block for one hour at RT on an orbital rotator. After centrifugation at 10,000 rpm for 5 minutes, the supernatant is aspirated. The pellet is resuspended manually with polyBSA solution (10 mg/ml). The particles are allowed to block for one hour at RT on an orbital rotator. After centrifugation at 10,000 rpm for 5 minutes, the supernatant is aspirated. The pellet is resuspended manually with acetylated or methylated BSA solution (10 mg/ml) to a final particle concentration of 1.0% solids.

To prepare the labeling zone solution, the labeling beads are diluted to a concentration of 0.1% solids in 10 mg/ml AcBSA or MBSA, prepared in 50 mM Tris buffer, pH 8.0, with 0.1% (w/v) NaN₃. Sucrose in 50 mM Tris buffer is added to a final concentration of 2.0%. The resultant mixture is stirred and dispensed onto Whatman F075-14 (Whatman, Inc., Fairfield, N.J.) material at 60 ul/cm². The material is frozen at −70° C. for at least one hour. Membranes are lyophilized in Virtis Genesis overnight. The label containing pads are cut into 3.5×3.0 mm rectangles and stored at less than 5.0% RH until assembly.

To prepare the capture zone membrane, nitrocellulose having a pore size of 8-12 um (Schleicher and Schuell) is affixed to an XY-plotter table. An hCG capture band is dispensed in a 2.0 mm zone at the distal end of the nitrocellulose membrane using Monoclonal Anti-hCG antibody (Clone MC097; Scripps Laboratories, San Diego, Calif.) at 1.0 mg/ml. The solution is dispensed with an IVEK Digispense dispensing system. After air drying at 45° C., the membrane is placed into a tray containing blocking solution (10 mg/ml AcBSA or mBSA) for 20 minutes at RT. The membrane is removed and blotted for 5 minutes. The membrane is air dried at 45° C. for 5 minutes, and then placed at less than 5.0% RH overnight. Processed capture membranes remain at less than 5.0% RH until assembly.

A 3.0×7.0 mm strip of the capture zone membrane is affixed centrally on an adhesive opaque strip. The opaque backing is a 350×23 mm strip of ARCare mylar made adhesive with 3M 9502.

The pad containing visible label is affixed next to the capture zone pad with 0.5 mm overlap. The sample treatment zone pad is then placed next to the label containing pad with 0.5 mm overlap.

The device is provided with an absorbent, which is a 3.5×3.0 mm rectangle of Whatman 31ET (Whatman, Inc.) membrane. It is placed distal to the capture membrane with 0.5 mm overlap.

The resultant test strip on the opaque backing is then placed membrane side down in the MP1 unit such that the sample treatment pad is overlapped by the sample receiving pad by 1.0 mm. The strip is aligned such that the label capturing zones on the capture membrane are visible through the optical aperture of the device. Finally, the top cover is placed together with the bottom casing such that the sample well is aligned over the sample receiving pad.

Biological samples including whole blood, plasma, serum, and urine were collected from healthy, asymptomatic donors. The specimens were analyzed on Dade Stratus® to determine endogenous hCG levels. Analyte was spiked into the specimens at varying concentrations and levels were confirmed on the Reference Quantitative Assay. Quantitative hCG values were assigned and the specimens were assayed as follows:

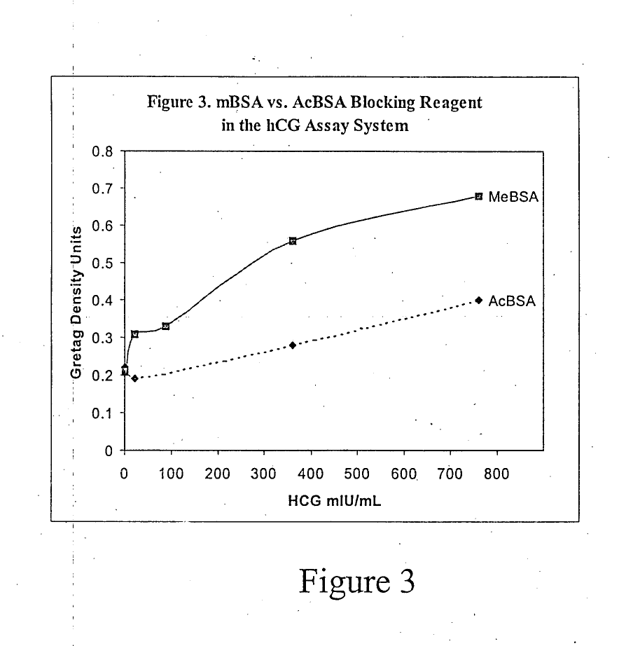

The device is placed flat on the bench top and 75 ul of sample is applied to the sample receiving zone. The liquid is allowed to flow through the four zones of the assay strip and collect in the absorbent pad. If hCG is present in the sample at least 25 mIU/mL, a blue band in the capture region will appear. The intensity of the band is measured with a Gretag reflectance densitometer. Increasing values from the Gretag indicate increasing color intensity. The performance of mBSA compared to AcBSA as a blocking reagent in the hCG assay system is shown in FIG. 3. The mBSA block gives much better curve separation than the AcBSA block.

Table 5 shows the assay results of 19 different male and female donors that are negative for hCG. None of the samples gave a false positive.

TABLE 5
Screening of hCG Negative Serum Samples

| Donor # | Sex | GDU | FP? |
|---|---|---|---|
| 180 | f | 0.20 | No |
| 181 | f | 0.20 | No |
| 184 | m | 0.20 | No |
| 185 | m | 0.20 | No |
| 186 | f | 0.25 | No |
| 187 | m | 0.20 | No |
| 188 | m | 0.28 | No |
| 189 | f | 0.21 | No |
| 190 | m | 0.20 | No |
| 179 | m | 0.21 | No |
| 183 | f | 0.22 | No |
| 191 | m | 0.22 | No |
| 192 | f | 0.22 | No |
| 193 | f | 0.21 | No |
| 194 | m | 0.19 | No |
| 195 | f | 0.20 | No |
| 196 | m | 0.20 | No |
| 197 | m | 0.21 | No |
| 198 | m | 0.20 | No |

Table 6 shows results of the screening of 14 different donors that tested positive for hCG by the Dade assay. All 14 samples also test positive by the Metrika assay.

TABLE 6
Screening of hCG Positive Serum Samples

| | |
|---|---|
| Metrika | Dade |

| Donor # | mIU/mL | Positive? | mIU/mL | Positive? |
|---|---|---|---|---|
| 2 | 4867 | Yes | 2965 | Yes |
| 5 | 5911 | Yes | 3011 | Yes |
| 6 | 1280 | Yes | 1699 | Yes |
| 9 | 6387 | Yes | 4905 | Yes |
| 52 | 7009 | Yes | 6113 | Yes |
| 64 | 7500 | Yes | 6646 | Yes |
| 96 | 346 | Yes | 689 | Yes |
| 82 | 4333 | Yes | 4051 | Yes |
| 87 | 4867 | Yes | 2740 | Yes |
| 83 | 382 | Yes | 220 | Yes |
| 19 | 127 | Yes | 70 | Yes |
| 97 | 155 | Yes | 86 | Yes |
| 91 | 461 | Yes | 196 | Yes |
| 24 | 491 | Yes | 274 | Yes |

### EXAMPLE 5

Alternate methods for preparation of the streptavidin capture zone for Troponin I assays were tested, utilizing application of biotinylated BSA to nitrocellulose followed by streptavidin solution.

Biotinylated BSA is prepared as follows. BSA is dissolved at 6 mg/ml in 1 ml of 0.1 M sodium phosphate buffer (pH 7.45) and 59 μl of sulfo-NHS-LC-biotin (EZ Link Sulfo-NHS-LC biotin; product #21335; Pierce Chemical Company) dissolved at 35 mg/ml of anhydrous DMF is added with stirring at RT. After 1 hr, the reaction mixture is buffer exchanged into 50 mM Tris/HCl buffer (pH 8.0) containing 0.1% (w/v) NaN₃ using PD-10 columns (code # 17-0851-01; Pharmacia Biotech AB, Uppsala, Sweden).

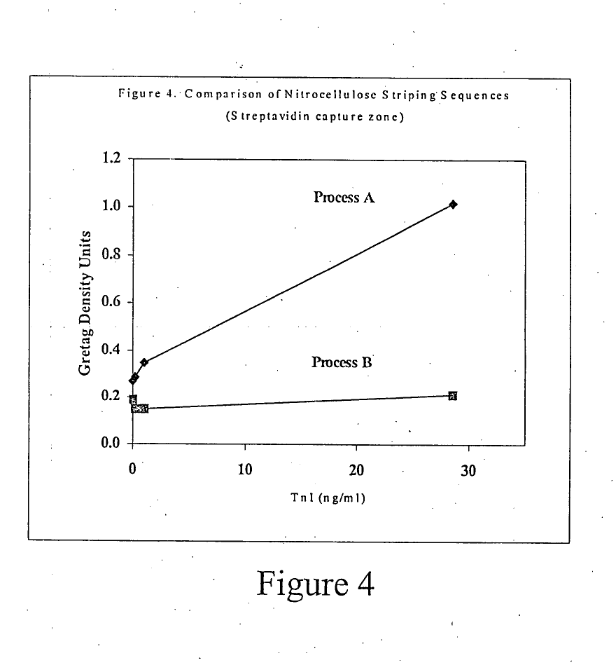

In the first process (refered to as Process A in FIG. 4 below), nitrocellulose having a pore size of >5 um (Schleicher and Schuell) is affixed to an XY table. A biotinylated BSA capture band is dispensed in a 2.0 mm zone at the distal end of the nitrocellulose membrane using biotinylated BSA at 2.0 mg/ml. The solution is dispensed with an IVEK Digispense dispensing system. The membrane is air dried at 45° C. for 15 min and placed into a tray containing the membrane blocking solution comprised of acetylated BSA solution at 10 mg/ml for 20 minutes at RT. The membranes are then removed and blotted for 5 minutes. The membranes are air dried at 45° C. for 15 minutes and streptavidin (code # SA10 010; Prozyme, Inc., San Leandro, Calif.) dissolved at 2 mg/ml in 50 mM Tris/HCl buffer (pH 8.0) containing 0.1% (w/v) NaN₃ is dispensed with an IVEK Digispense dispensing system. The membranes are then air dried at 45° C. for 15 minutes, washed for 5 min with 50 mM Tris/HCl buffer (pH 8.0) containing 0.1% (w/v) NaN₃ Finally, the membranes are placed again into a tray containing the membrane blocking solution comprised of acetylated BSA solution at 10 mg/ml for 20 minutes at RT. The membranes are then removed and blotted for 5 minutes. The membranes are air dried at 45° C. for 15 minutes and then placed at less than 5.0% RH overnight. Processed capture membranes remain at less than 5.0% RH until assembly.

In the second process (refered to as Process B in FIG. 4 below), nitrocellulose having a pore size of >5 um (Schleicher and Schuell) is affixed to an XY table. A biotinylated BSA capture band is dispensed in a 2.0 mm zone at the distal end of the nitrocellulose membrane using biotinylated BSA at 2.0 mg/ml. The solution: is dispensed with an IVEK Digispense dispensing system. The membrane is air dried at 45° C. for 15 min and streptavidin dissolved at 2 mg/ml in 50 mM Tris/HCl buffer (pH 8.0) containing 0.1% (w/v) NaN₃ is dispensed with an IVEK Digispense dispensing system. The membranes are then air dried at 45° C. for 15 minutes, washed for 5 min with 50 mM Tris/HCl buffer (pH 8.0) containing 0.1% (w/v) NaN₃ and placed into a tray containing the membrane blocking solution comprised of acetylated BSA solution at 10 mg/ml for 20 minutes at RT. The membranes are then removed and blotted for 5 minutes. The membranes are air dried at 45° C. for 15 minutes and then placed at less than 5.0% RH overnight. Processed capture membranes remain at less than 5.0% RH until assembly.

To activate the labeling beads, one ml of 0.403 um Dark Blue latex particles at 10% (w/v) solids are combined with 1 ml of 0.5 M MES buffer (pH 6.0), 5.5 ml of deionized H₂O, 2.3 ml of 50 mg of NHS per ml deionized H₂O and 0.2 ml of 5 mg of EDC in deionized H₂O. The resultant mixture is sonicated on ice for 40 sec and then allowed to react on a shaker at RT for 30 min. The activated MPs are then centrifuged at 10° C. at 10,000×g and washed three times with cold 50 mM MES buffer (pH 6.0) by resuspension and centrifugation cycles. In a typical procedure, the final pellet of MPs is suspended in 3.666 ml of 50 mM MES buffer (pH 6.0), a 5 ml mixture of 0.6-1.2 mg antigen-specific antibody such as e.g. goat anti-Troponin I peptide 3 antibody (Product code # G-129-C; BiosPacific/Fortron Bio Science Inc.) and 2.5 mg of mouse IgG in the same buffer is added with mixing, followed by an addition of 5 ml of 0.1 M borate buffer (pH 8.5). The mixture is allowed to incubate at RT for 2 hr and then centrifuged as described above. Subsequently, 10 ml of 50 mM borate buffer (pH 8.5) containing 5 mM ethanolamine is added to the pellet, MPs are suspended, incubated at RT for 30 min, and the suspension is centrifuged as described above. The remaining hydrophobic sites on MPs are then blocked with acetylated BSA solutions (10 mg/ml) described above; the pellet is resuspended to a final particle concentration of 0.5% (w/v) solids.

For “wet” assays for Troponin I, a 14×100 mm strip of the capture zone membrane is affixed centrally on an adhesive opaque strip. The opaque backing is a 23×100 mm strip of ARCare mylar made adhesive with 3M 9502. The absorbent—which is a 10×100 mm rectangle of Whatman 31ET cellulose paper—is affixed distal to the capture zone pad with 0.5 mm overlap. The sample zone pad composed of 7×100 mm cellulose acetate is then placed next to the capture zone membrane with 0.5 mm overlap.

Serum samples were drawn at Metrika, Inc. from apparently healthy asymptomatic volunteers. The specimens were then analyzed by Behring Opus Plus Troponin I Reference Quantitative Assay and/or by Dade Stratus® Cardiac Troponin-I Fluorometric Enzyme Assay, and they were found to be negative for Troponin I. Subsequently, a Troponin I+C complex (cat. # T5124; Scripps Laboratories) was used as an exogenous source of Troponin I which was added to the serum specimens.

Ten-fold concentrated STB for “wet” assays comprised of 0.5 M Tris buffer (pH 8.0) supplemented with 10 mg/ml of acetylated BSA, 0.3-0.6 mg/ml of heterophilic IgG block Heteroblock; PIN 70506; Omega Biologicals Inc., Bozeman, Mont.), 0.1% (w/v) of NaN₃, 0.5 M Urea, and 5% (v/v) of bovine serum (cat. # B 8655; Sigma Chemical Company).

In “wet” assays, 10 μl of specimen sample is mixed sequentially in a test tube with 3.4 μl of 10-fold concentrated stock of STB, 1 μl of biotinylated F(ab′)₂ fragment of goat anti-TnI

| CLAIM | RECITAL | SUPPORT |
|---|---|---|
| 2 | A method of preparing at least one surface | The assays described in the |
| | | specification utilize “particles” |
| | | (page 2, line 19), and |
| | | “membrane[s]” (page 3, line 14), |
| | | with treated surfaces. |
| | To reducereduced nonspecific interference | The specification recites the |
| | | elimination of “non-specific |
| | | interference with the detection |
| | | agents and/or binding partner |
| | | caused by endogenous polypeptide |
| | | constituents” (page 2, lines 5 to 6). |
| | for use in an immunoassay comprising the steps | The title of the application is |
| | of: | “Blocking Compositions for |
| | | Immunoassays.” |
| | a) coating all or part of the surface with a | The “binding partner” is recited on |
| | binding partner; and | page 1, line 11, and coating of the |
| | | surface of a membrane or particle |
| | | with the binding partner is |
| | | exemplified by antibodies (page 2, |
| | | lines 29-30) and streptavidin (page |
| | | 3, line 16). |
| | b) blocking the surface with anhydride-, | “Chemically modified albumins” |
| | alkyl acetimidate- or aldehyde-treated albumin. | are described (page 1, line 13) in |
| | | which the modification is: |
| | | anhydride treatment (page 5, line 6 |
| | | to page 6, line 20); alkyl |
| | | acetimidate treatment (page 6, lines |
| | | 21 to 29); or aldehyde treatment |
| | | (page 7, lines 1 to 15). |
| 3 | The method according to claim 2, wherein the | The specification recites |
| | immunoassay is an immunochromatographic | “immunochromatographic assays” |
| | assay. | on page 1, line 8. |
| 4 | The method according to claim 3, wherein the | The specification recites “lateral |
| | immunochromatographic assay is a lateral flow | flow immunochromatographic |
| | assay. | assays” on page 1, line8. |
| 5 | The method according to claim 2, wherein the | The specification recites coating a |
| | surface is a membrane. | “capture zone membrane” with a |
| | | binding partner and albumin on |
| | | page 3, lines 14 to 26. |
| 6 | The method according to claim 5, wherein the | The specification recites affixing |
| | membrane is nitrocellulose. | nitrocellulose to prepare the capture |
| | | zone membranes, on page 3, lines |
| | | 14 to 15. |
| 7 | The method according to claim 2, wherein the | The specification describes assays |
| | surface is a particle. | utilizing “particles” on page 2, line |
| | | 19. |
| 8 | The method according to claim 7, wherein the | The specification discloses“latex |
| | particle is latex. | particles” on page 2, line 19. |
| 9 | The method according to claim 2, wherein the | The specification describes coating |
| | binding partner is an antibody. | surfaces such as particles with |
| | | antibodies on page 2, lines 28 to |
| | | 29. |
| 10 | The method according to claim 2, wherein the | The specification recites coating |
| | binding partner is serum streptavidin-bovine | surfaces such as membranes with |
| | serum albumin conjugate. | streptavidin-bovine serum albumin |
| | | on page 3, lines 14 to 18. |
| 11 | The method according to claim 2, wherein the | The specification recites treatment |
| | albumin is anhydride-treated. | of albumins with anhydrides on |
| | | page 5, line 6 to page 6, line 20. |
| 12 | The method according to claim 11, wherein the | The specification recites that the |
| | anhydride is selected from the group consisting | anhydride can be: maelic |
| | of maleic anhydride, acetic anhydride, or | anhydride (page 5, line 7); acetic |
| | succinic anhydride. | anhydride (page 6, line 5); or |
| | | succinic anhydride (page 6, line |
| | | 13). |
| 13 | The method according to claim 2, wherein the | The specification recites aldehyde |
| | albumin is aldehyde-treated. | treatment on page 7, lines 1 to 15. |
| 14 | The method according to claim 13, wherein the | Exemplary aldehydes are: |
| | aldehyde is glutaraldehyde or formaldehyde. | “glutaraldehyde” which is recited in |
| | | the specification on page 7, line 11; |
| | | and “formaldehyde” which is |
| | | recited in the specification on page |
| | | 7, line 2. |
| 15 | The method according to claim 2, wherein the | The specification recites alkyl |
| | albumin is alkyl acetimidate-treated. | acetimidate treatment on page 6, |
| | | lines 21 to 29. |
| 16 | The method according to claim 15, wherein the | The specification describes the |
| | alkyl acetimidate is methyl acetimidate or ethyl | exemplary alkyl acetimidates |
| | acetimidate. | “methyl or ethyl acetimidates” on |
| | | page 6, line 21. |
| 17 | The method according to claim 2, wherein the | The specification describes assays |
| | immunoassay further comprises an | utilizing “particles” on page 2, line |
| | immunochromatographic assay performed on a | 19. |
| | membrane utilizing particles, | |
| | wherein step a) further comprises coating all or | The specification recites coating |
| | part of the surface of both the membrane and | both the membrane and the |
| | the particles with a binding partner; | particles with a binding partner on |
| | | page 12, line 10 to page 13, line |
| | | 18.. |
| | and wherein step b) further comprises blocking | The specification recites blocking |
| | the surface of both the membrane and the | the surface of both the membrane |
| | particles with anhydride-, alkyl acetimidate-, or | and the particles on page 12, line |
| | aldehyde-treated albumin. | 10 to page 13, line 18. |
| 18 | The method according to Claim 2, wherein the | The specification recites processes |
| | immunoassay is adapted to detect an analyte in | for assaying “analytes in body |
| | a body fluid, | fluids” on page 2, lines 3 to 4. |
| | and wherein the body fluid is treated with | The specification recites |
| | anhydride-, alkyl acetimidate- or aldehyde- | pretreatment of the body fluids |
| | treated albumin prior to contacting the surface. | with, for example, acetylated |
| | | bovine serum albumin on page 14, |
| | | lines 12 to 19. |
| 19 | A method of preparing an immunoassay system | The specification describes treating |
| | for detecting an analyte in a body fluid, | a membrane surface or particle, |
| | comprising the steps of: | and a body fluid with for example, |
| | a) coating a membrane surface with a first | acetylated albumin on page 12, line |
| | binding partner; | 10 to page 14, line 11. |
| | b) coating a particle with a second binding | |
| | partner; and | |
| | c) treating the membrane, the particle and the | |
| | body fluid with anhydride-, alkyl acetimidate-, | |
| | or aldehyde- treated albumin. | |

1. (canceled)

2. A method of preparing at least one surface to reduce nonspecific interference for use in an immunoassay comprising the steps of:
a) coating all or part of the surface with a binding partner; and
b) blocking the surface with anhydride-, alkyl acetimidate- or aldehyde-treated albumin.

3. The method according to claim 2, where the immunoassay is immunochromatographic assay.

4. The method according to claim 2, where the immunochromatographic assay is a lateral flow assay.

5. The method according to claim 2, wherein the surface is a membrane.

6. The method according to claim 5, wherein the membrane is nitrocellulose.

7. The method according to claim 2, wherein the surface is a particle.

8. The method according to claim 7, wherein the particle is a latex particle.

9. The method according to claim 2, wherein the binding partner is an antibody.

10. The method according to claim 2, wherein the binding partner is a streptavidin-bovine serum albumin conjugate.

11. The method according to claim 2, where the albumin is anhydride-treated.

12. The method according to claim 11, wherein the anhydride is selected from the group consisting of maleic anhydride, acetic anhydride and succinic anhydride.

13. The method according to claim 2, wherein the albumin is aldehyde-treated.

14. The method according to claim 13, wherein the aldehyde is glutaraldehyde or formaldehyde.

15. The method according to claim 2, wherein the albumin is alkyl acetimidate-treated.

16. The method according to claim 15, wherein the acetimidate is methyl acetimidate or ethyl acetimidate.

17. The method according to claim 2, wherein the immunoassay further comprises an immunochromatographic assay performed on a membrane utilizing particles, wherein step a) further comprises coating all or part of the surface of both the membrane and the particles with a binding partner; and wherein step b) further comprises blocking the surface of both the membranes and the particles with anhydride-, alkyl acetimidate-, or aldehyde-treated albumin.

18. The method according to claim 2, wherein the immunoassay is adapted to detect an analyte in a body fluid, and wherein the body fluid is treated with anhydride-, alkyl acetimidate- or aldehyde-treated albumin prior to coming in contact with the surface.

19. (canceled)
